(19) United States
(12) Patent Application Publication
(10) Pub. No.: US 2026/0200297 A1
(43) Pub. Date: Jul. 16, 2026

(54) Garment-shaped Vehicle Air Freshener

(71) Applicant: Kevin Damron, Yuba City, CA (US)

(72) Inventor: Kevin Damron, Yuba City, CA (US)

(21) Appl. No.: 19/024,328

(22) Filed: Jan. 16, 2025

(51) Int. Cl. B60H 3/00; A61L 9/01; A61L 9/12; A61L 101/32; A61L 101/34; A61L 101/36; A61L 101/44

(52) U.S. Cl. CPC B60H 3/0014; A61L 9/01; A61L 9/12; A61L 2101/32; A61L 2101/34; A61L 2101/36; A61L 2101/44; A61L 2209/15

(57) ABSTRACT

A novelty air freshener device configured in the shape of women's undergarments, featuring a porous body for gradual fragrance release. The air freshener incorporates adult-oriented fragrances, including formulations designed to replicate pheromonal or musk-like scents, creating an innovative and marketable product for enhancing vehicle ambiance. The design includes decorative and functional elements such as cut-outs, an aperture for mounting, and the capacity for seasonal and style variations.

### CROSS-REFERENCE TO RELATED APPLICATIONS

There are no previously filed, nor currently any co-pending applications, anywhere in the world.

### BACKGROUND OF THE INVENTION

### 1. Field of the Invention

The present invention relates generally to decorative air freshening devices for vehicles, and more particularly to a novelty air freshener having an ornamental design resembling women's undergarments and incorporating specialized fragrances intended to enhance vehicle ambiance.

### 2. Description of the Related Art

Vehicle air fresheners have long been used to mask unpleasant odors and provide pleasant fragrances within automobile cabins. Traditional designs have typically included simple geometric shapes, trees, or other common decorative forms. Within the automotive accessory market, there exists a significant segment of consumers who seek to personalize their vehicles with novelty items that reflect their individual tastes and preferences.

The adult novelty market has demonstrated consistent demand for automotive accessories that incorporate mature themes or suggestive designs. However, existing air freshener products in this category have generally been limited to conventional shapes with basic graphic designs or text. There remains an unfulfilled market need for air fresheners that combine functional odor control with more sophisticated adult-oriented design elements that can enhance vehicle ambiance while maintaining aesthetic appeal.

Additionally, while various fragrances have been developed for automotive air fresheners, these have typically focused on common scents such as pine, vanilla, or “new car” smell. There exists a gap in the market for specialized scent profiles that specifically target adult-oriented themes and experiences, particularly those attempting to replicate human pheromones or similar attractive scents.

Some methods and devices are known that incorporate various mechanisms for identifying the movement of mobile phones, or for otherwise limiting some of their capabilities. For example:

U.S. Pat. No. D 1,261,196 entitled Air Freshener for Vehicles shows a design patent for a vehicle air freshener, focusing on the visual appearance of the product.

U.S. Patent Publication No. 2018/0056757, entitled USB Plug-In Vehicle Air Freshener, describes a USB plug-in vehicle air freshener that dispenses fragrance through a heating element and can be controlled wirelessly.

U.S. Patent No. D 704,322, entitled Vehicle Air Freshener, shows another design patent for a vehicle air freshener, highlighting the aesthetic aspects of the product.

And again, U.S. Patent No. D 596,282, entitled Decorative Motor Vehicle Air Freshener, provides a decorative air freshener for vehicles, emphasizing the design and visual appeal.

Furthermore, current adult-themed automotive accessories often lack the ability to be customized for different seasons or holidays, limiting their market appeal and potential for repeat customer engagement. A need exists for a product line that can be regularly refreshed with new designs while maintaining a consistent brand identity within this specific market niche.

This invention aims to address these market gaps by providing a novel approach to vehicle air fresheners that combines suggestive design elements with practical functionality, while establishing a platform for potential expansion into seasonal variations and specialized scent profiles.

### SUMMARY OF THE INVENTION

The object of the present invention is to provide a novelty vehicle air freshener that combines the functional aspects of scent dispersal with adult-oriented decorative design elements shaped like women's undergarments, thereby creating a distinct market offering in automotive accessories.

The present invention is characterized by an air freshener comprising a flat, porous paperboard body having first and second sides configured to depict front and rear views of a women's undergarment, specifically a thong design, wherein an upper portion represents a waist area including cut-out portions depicting a low-cut front area and a central portion representing a rear strap, and a lower portion depicting a crotch area, with an aperture located along a bisecting axial centerline adjacent the upper edge for mounting purposes.

This main feature captures the core novel aspect-the specific ornamental configuration of the air freshener-while including the basic functional elements of construction and mounting. The specialized fragrance aspect, while mentioned in the documents, appears secondary since it is not yet fully developed or disclosed.

It is an advantage of the present invention to provide a decorative air freshener that combines functional fragrance dispersal with adult-oriented design elements.

It is another advantage of the present invention to provide an air freshener fabricated from porous, absorbent paperboard material capable of holding and gradually releasing fragrance over an extended period.

It is another advantage of the present invention to provide an air freshener having a design that implies a woman's undergarment with distinct front and rear visual elements.

Another advantage of the present invention is to provide an air freshener that can be easily mounted within a vehicle cabin through a simple hanging mechanism.

It is another advantage of the present invention to provide a design platform capable of supporting multiple style variations and seasonal editions.

It is another advantage of the present invention to provide a resilient construction that withstands handling and environmental concerns while maintaining fragrance integrity during storage.

It is another advantage of the present invention to provide an air freshener capable of incorporating specialized scents that mimic female pheromones to enhance attraction, mood, or ambiance.

It is another advantage of the present invention to provide a novel approach to automotive accessories that targets the adult-oriented product market segment.

Further features of the invention will become apparent in the course of the following description.

### BRIEF DESCRIPTION OF DRAWINGS

The advantages and features of the present invention will become better understood with reference to the following more detailed description and claims taken in conjunction with the accompanying drawings, in which like elements are identified with like symbols, and in which:

FIG. 1 is a first side isometric view of an air freshener 10, according to a preferred embodiment of the present invention;

FIG. 2 is a second side isometric view of the air freshener 10, according to the preferred embodiment of the present invention; and,

FIG. 3 is an environmental view of the air freshener 10 mounted within a cabin 32 of a vehicle 35, according to an exemplary method of use of the present invention.

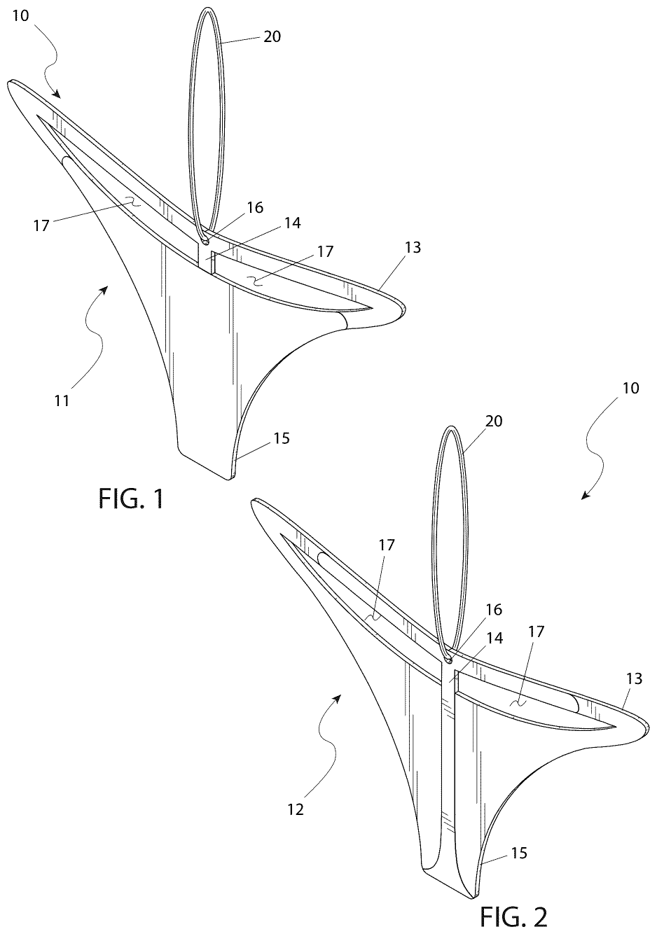

### DETAILED DESCRIPTION OF THE INVENTION

The best mode for carrying out the invention is presented in terms of its preferred embodiment, herein depicted within the Figures. It should be understood that the legal scope of the description is defined by the words of the claims set forth at the end of this patent and that the detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. § 112(f).

The best mode for carrying out the invention is presented in terms of its preferred embodiment, herein depicted within the Figures.

### 1. Detailed Description of the Figures

Referring now to the drawings, wherein like reference numerals indicate the same parts throughout the several views, an air freshener 10 is shown according to the preferred embodiment of the present invention. The air freshener 10 is capable of emitting a fragrance that is embedded, impregnated, or otherwise present. The air freshener 10 preferably comprises a first side 11 and a second side 12, as well as an upper portion 13 and a lower portion 15. The air freshener 10 can be fabricated out of a paperboard designed to be porous and absorbent, thereby allowing it to hold and gradually release the fragrance over a specified period of time and also resilient due to handling and environmental concerns. The air freshener 10 may be packaged in such a way as to enable lengthy storage prior to use without damage or dissipation of the fragrance.

In an exemplary embodiment, the first side 11 and second side 12 preferably comprises indicia or a form that implies a woman's undergarment (e.g. a thong), such that the first side 11 depicts a front side thereof and the second side 12 depicts a rear side thereof. The upper portion 13 depicts the waist area of the undergarment, and the lower portion 15 depicts the crotch area of the undergarment. The upper portion 13 further comprises a pair of cut-out portions 17. In the exemplary embodiment, the pair of cut-out portions 17 represent a low-cut area of a front side of the undergarment. The upper portion 13 also comprises a central portion 14 that bisects the pair of cut-out portions 17. The central portion 14 represents the rear strap on a rear side of an undergarment.

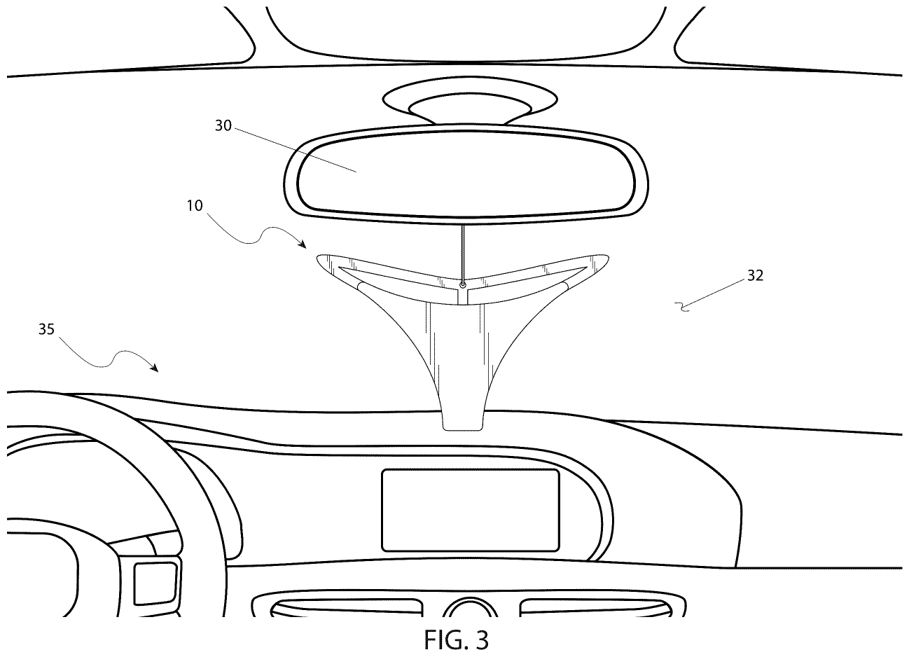

An aperture 16 is located along a bisecting axial centerline adjacent to the upper edge of the upper portion 13. The aperture 16 is large enough to permit the passage of a mounting means 20, such as a string, in order to suspend the air freshener 10 from a desired location. FIG. 3 depicts an exemplary method of use, where the air freshener 10 is suspended from a rearview mirror 30 on a cabin 32 of a vehicle 30, although other locations and mounting means 20 may be used to fall under the overall scope of the invention. In an exemplary embodiment, the fragrance may mimic female pheromones in an attempt to correspond to the undergarment indicia of the air freshener 10. The use of a fragrance that mimics female pheromones may be essentially suited for marketing adult-oriented products or experiences in order to enhance attraction, mood, or ambiance.

In a further embodiment, the air freshener's fragrance compound can include formulations designed to emulate the scent of female vaginal discharge. This may be achieved using compounds that replicate certain pheromonal or musk-like notes. Ingredients such as civetone (a synthetic musk), androsterone (a natural pheromone), or combinations of lactonic compounds, fatty acids, and sulfur-containing molecules known to contribute to such olfactory profiles, may be incorporated. For the purpose of emulating the smell of female vaginal discharge, the following compounds may further be included:

Trimethylamine: This is the primary cause of the fishy odor associated with bacterial vaginosis.

Putrescine, Cadaverine, and Tyramine: These amines are also present in high concentrations in vaginal fluid from women with bacterial vaginosis, contributing to the malodorous discharge.

Acetic Acid: This compound can be present in vaginal fluid and may contribute to a sour or tangy smell, although it is more commonly associated with gynecological tumors and other conditions.

Further still, to emulate the smell of female vaginal discharge, the following compounds may also be considered:

For a healthy vaginal smell, compounds that mimic the sour or tangy aroma of Lactobacilli, such as lactic acid or other organic acids found in fermented foods.

For an abnormal smell, such as that associated with BV, trimethylamine and other amines like putrescine, cadaverine, and tyramine could be used.

These compounds, individually or in combination, may be selected and synthesized to provide a distinctive scent that aligns with the intended adult-oriented market. The inclusion of such fragrances offers an innovative approach to creating an ambient vehicle experience that appeals to specific consumer preferences.

### 2. Operation of the Preferred Embodiment

In operation, the garment-shaped air freshener is designed to be easily suspended within a vehicle cabin using a mounting means, such as a string or hook, threaded through the aperture located on the upper portion of the device. Once mounted, the porous, absorbent material of the air freshener begins to release the embedded fragrance compound gradually into the surrounding environment. The fragrance compound, which may include specialized adult-oriented or pheromonal scents, disperses evenly over time, providing a sustained olfactory experience that enhances the vehicle's ambiance. The air freshener's novelty design, resembling women's undergarments, adds a decorative and suggestive aesthetic that appeals to the adult-themed market, while the fragrance's unique profile caters to sensory preferences, mood enhancement, and personal expression. The device is constructed to maintain its structural integrity and fragrance potency during storage and use, ensuring a long-lasting and impactful experience for consumers.

The foregoing descriptions of specific embodiments of the present invention are presented for purposes of illustration and description. The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples, and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way. Any unintended embracement of such subject matter is hereby disclaimed. They are not intended to be exhaustive nor to limit the invention to precise forms disclosed and, obviously, many modifications and variations are possible in light of the above teaching. The embodiments are chosen and described in order to best explain principles of the invention and its practical application, to thereby enable others skilled in the art to best utilize the invention and its various embodiments with various modifications as are suited to the particular use contemplated. It is intended that a scope of the invention be defined broadly by the Drawings and Specification appended hereto and to their equivalents. Therefore, the scope of the invention is in no way to be limited only by any adverse inference under the rulings of Warner-Jenkinson Company, v. Hilton Davis Chemical, 520 US 17 (1997) or Festo Corp. v. Shoketsu Kinzoku Kogyo Kabushiki Co., 535 U.S. 722 (2002), or other similar caselaw or subsequent precedent should not be made if any future claims are added or amended subsequent to this Patent Application.

1. A decorative air freshener device comprising:
a flat, porous body having first and second sides configured to visually represent front and rear views of a women's undergarment;
an upper portion comprising a pair of cut-out portions and a central bisecting portion;
a lower portion;
an aperture located along a bisecting axial centerline adjacent said upper portion; and
a fragrance compound embedded within said porous body comprising at least one compound selected from the group consisting of civetone, androsterone, lactic acid, trimethylamine, putrescine, cadaverine, tyramine, and acetic acid.

2. The device of claim 1, wherein said porous body comprises an absorbent paperboard material.

3. The device of claim 1, further comprising a mounting means inserted through said aperture.

4. The device of claim 1, wherein said women's undergarment shape comprises a thong design.

5. The device of claim 1, wherein said central bisecting portion represents a rear strap of said undergarment.

6. The device of claim 1, wherein said fragrance compound further comprises lactonic compounds and fatty acids.

7. A method of manufacturing a decorative air freshener comprising:
forming a porous substrate into a shape resembling women's undergarments;
incorporating visual indicia on first and second sides representing front and rear views of said undergarments;
creating an aperture for mounting purposes;
preparing a fragrance formulation comprising at least one compound selected from the group consisting of civetone, androsterone, lactic acid, trimethylamine, putrescine, cadaverine, tyramine, and acetic acid; and
impregnating said substrate with said fragrance formulation.

8. The method of claim 7, wherein said fragrance formulation further comprises at least one musk compound.

9. The method of claim 7, wherein said fragrance formulation comprises a combination of at least one musk compound and at least one amine compound.

10. A vehicle air freshener system comprising:
a decorative body shaped as women's undergarments and adapted for mounting said body within a vehicle cabin;
a fragrance delivery mechanism comprising a porous substrate; and
a fragrance compound comprising:
(a) at least one musk compound selected from the group consisting of civetone and androsterone;
(b) at least one acid compound selected from the group consisting of lactic acid and acetic acid; and
(c) at least one amine compound selected from the group consisting of trimethylamine, putrescine, cadaverine, and tyramine.

11. The system of claim 13, wherein said fragrance delivery mechanism comprises said porous substrate material impregnated with said fragrance compound.

12. The system of claim 13, wherein said decorative body includes distinctive front and rear visual elements representing a thong-style undergarment.

13. (canceled)
